(19) United States
(12) Patent Application Publication
(10) Pub. No.: US 2026/0325420 A1
(43) Pub. Date: Oct. 22, 2026

(54) METHOD OF REDUCING PIGMENTATION AND SKIN-DARKENING IN MELANIN-RICH SKIN CAUSED BY SOLAR RADIATION

(71) Applicant: BEIERSDORF AG, Hamburg (DE)

(72) Inventors: Eduardo C. RUVOLO, Flanders, NJ (US); Priyanka PUJARI, Mumbai (IN); Koushik SREEDHAR, Mumbai (IN); Sonali KULKARNI, Nashik (IN); Veena BELHEKAR, Mumbai (IN)

(21) Appl. No.: 19/181,624

(22) Filed: Apr. 17, 2025

(51) Int. Cl. A61K 8/37; A61Q 17/04; A61Q 19/02

(52) U.S. Cl. CPC A61K 8/37; A61Q 17/04; A61Q 19/02; A61K 2800/5922

(57) ABSTRACT

A method of reducing pigmentation, skin-darkening and erythema caused by solar radiation in melanin-rich skin comprises applying to skin in need thereof a cosmetic composition which comprises Pentaerythrityl Tetra-di-t-butyl Hydroxyhydrocinnamate and/or Diethylhexyl Syringylidene Malonate.

### FIELD OF THE INVENTION

The present invention relates a method of reducing pigmentation, skin-darkening and erythema caused by solar radiation in melanin-rich skin and to a composition which is suitable for use in this method.

### BACKGROUND OF THE INVENTION

The human skin serves as the body's first line of defense against extrinsic factors aggressors such as ultraviolet (UV) radiation, pollution, and oxidative stress. Exposure to UV radiation is the most common, and also the most preventable, cause of skin cancers. Skin damage with UV exposure occurs through at least two mechanisms: (i) direct damage to DNA, mainly from UVB (280 to 320 nm), and (ii) production of free radicals from UVA (320 to 400 nm), which destroy intracellular components including DNA, membranes, and proteins. Cosmetic products with protection against UV, which are commonly marketed as gel, cream, emulsion or spray aim to protect the skin of a person from the rays emitted by the sun.

However, existing sunscreen formulations have historically been designed for lighter skin types, often leaving visible residues, or inadequately addressing the needs of melanin-rich skin. Melanin-rich skin, commonly classified as Fitzpatrick skin types IV-VI, has unique physiological characteristics, including higher melanin content, which offers some natural protection against UV damage. Nonetheless, it is not immune to the harmful effects of UV radiation.

The key challenges posed by melanin-rich skin are: (i) hyperpigmentation and skin darkening where UVA, UVB and Visible Light (VL) can trigger excessive melanin production, leading to uneven skin tone and dark spots, (ii) post-inflammatory hyperpigmentation which causes the skin to discolor following UVA, UVB and VL exposure or inflammation, and (iii) compatibility issues that result in a visible white deposit on the skin when an effective amount of cosmetic sunscreen composition is applied to melanin-rich skin.

Pigmentary changes caused by VL and UVA have been shown to occur in three phases, i.e., immediate pigment darkening (IPD) which is dose dependent and lasts up to 2 hours after irradiation, followed by persistent pigment darkening (PPD) that continues up to 24 hours, and lastly delayed tanning (DT) which occurs approximately 7 days after irradiation. Both IPD and PPD have been suggested to be a result of oxidation and redistribution of pre-existing melanin, whereas delayed tanning is caused by the formation of new melanin.

Despite the fact that VL and UVA have been shown to be associated with pigment darkening and worsening conditions such as post-inflammatory hyperpigmentation and melasma, there are limited options available for alleviating these conditions. Furthermore, there is a lack of formulations specifically tailored to melanin-rich skin, which requires products that not only provide broad-spectrum UV protection but also prevent skin darkening and promote skin health. Accordingly, there remains a need for more effective sunscreen compositions, and particularly formulations that can provide effective protection from solar radiation while minimizing skin darkening and promoting skin health for skin of color.

In view of the foregoing, it would be advantageous to be able to provide extended protection of melanin-rich skin against damage caused by solar radiation, especially in order to suppress pigmentation and erythema induced by solar radiation in the UVB, UVA, and VL ranges.

The inventors have found that solving the problem of regulating the behavior of melanin-rich skin caused by solar radiation is complicated but very important, in particular for formulating products for application to melanin-rich skin with a view to ensuring a satisfactory protection against skin darkening and erythema.

### SUMMARY OF THE INVENTION

The present invention provides a method of reducing pigmentation, skin-darkening and erythema caused by solar radiation in melanin-rich skin. The method comprises applying to skin in need thereof a cosmetic composition comprising Pentaerythrityl Tetra-di-t-butyl Hydroxyhydrocinnamate and/or Diethylhexyl Syringylidene Malonate in an amount which is effective for reducing pigmentation, skin darkening and erythema caused by solar radiation in melanin-rich skin.

In one embodiment of the method, the method may be a therapeutic method. In another embodiment of the method, the method may be a cosmetic or non-therapeutic method.

In one embodiment of the method, the composition comprises at least Pentaerythrityl Tetra-di-t-butyl Hydroxyhydrocinnamate or comprises at least Diethylhexyl Syringylidene Malonate. In one embodiment of the method, the composition comprises both Pentaerythrityl Tetra-di-t-butyl Hydroxyhydrocinnamate and Diethylhexyl Syringylidene Malonate.

For example, the composition may comprise Pentaerythrityl Tetra-di-t-butyl Hydroxyhydrocinnamate and/or Diethylhexyl Syringylidene Malonate in a total concentration of from 0.05% to 2% by weight, based on the total weight of the composition.

In one embodiment of the method, the composition comprises Pentaerythrityl Tetra-di-t-butyl Hydroxyhydrocinnamate in a concentration of from 0.1% to 1% by weight, e.g., from 0.5% to 0.8% by weight, and/or comprises Diethylhexyl Syringylidene Malonate in a concentration of from 0.1% to 1% by weight, e.g., from 0.5% to 0.8% by weight, each based on a total weight of the composition.

In one embodiment of the method, the composition further comprises one or more UV filter substances, preferably only organic UV filter substances. For example, the one or more UV filter substances may comprise at least one, at least two or all of Bis-Ethylhexyloxyphenol Methoxyphenyl Triazine, Butyl Methoxydibenzoylmethane, and Ethylhexyl Salicylate.

In one embodiment of the method, the composition comprises the one or more UV filter substances in a total concentration of from 1% to 20% by weight, e.g., from 5% to 15% by weight, based on the total weight of the composition.

In one embodiment of the method, oxy-hemoglobin levels are reduced.

The present invention further provides a composition which is formulated for topical application to skin. The composition comprises Pentaerythrityl Tetra-di-t-butyl Hydroxyhydrocinnamate and/or Diethylhexyl Syringylidene Malonate in a concentration which is effective for reducing pigmentation, skin darkening and erythema caused by solar radiation in melanin-rich skin when applied to skin in need thereof. The composition may be suitable for use in the method of the invention as set forth above (including the various embodiments thereof).

In one embodiment, the composition comprises Pentaerythrityl Tetra-di-t-butyl Hydroxyhydrocinnamate and/or Diethylhexyl Syringylidene Malonate in a total concentration of from 0.05% to 2% by weight, e.g., from 0.1 to 1% by weight, based on the total weight of the composition.

In one embodiment, the composition further comprises one or more UV filter substances, e.g., one or more organic UV filter substances. For example, the one or more UV filter substances may comprise at least one of Bis-Ethylhexyloxyphenol Methoxyphenyl Triazine, Butyl Methoxydibenzoylmethane, and Ethylhexyl Salicylate. Further non-limiting examples of suitable UV filter substances comprise para-aminobenzoic acid, avobenzone, cinoxate, dioxybenzone, homosalate, menthyl anthranilate, octocrylene, octyl methoxycinnamate, octyl salicylate, oxybenzone, padimate O, phenylbenzimidazole sulfonic acid, octisalate, sulisobenzone, trolamine salicylate, titanium dioxide and zinc oxide, diethanolamine methoxycinnamate, digalloyl trioleate, ethyl dihydroxypropyl PABA, glyceryl aminobenzoate, lawsone with dihydroxy acetone, red petrolatum, 4-Methylbenzylidene camphor, Bis-Ethylhexyloxyphenol Methoxyphenyl Triazine, Methylene Bis-Benzotriazolyl Tetramethylbutylphenol, Polysilicone-15. Bis-Ethylhexyloxyphenol Methoxyphenyl Triazine, Butyl Methoxydibenzoylmethane, and Ethylhexyl Salicylate.

In one embodiment, the composition comprises the one or more UV filter substances in a total concentration of from 1% to 30% by weight, e.g., 5% to 20% by weight, based on the total weight of the composition.

In one embodiment, the composition is present in the form of an emulsion (e.g., an O/W emulsion or a W/O emulsion) and/or the in the form of a gel, a cream, a lotion or a spray. The composition is a topical composition and/or a cosmetic composition.

If the composition of the invention comprises Pentaerythrityl Tetra-di-t-butyl Hydroxyhydrocinnamate, the composition is free of inorganic UV filter substances and is not suitable for use as, e.g., antiperspirant, deodorant or personal care composition. For example, the composition does not comprise an antiperspirant active. The composition also is free from magnolia extracts, zinc pyrithione, a thiopyridinone compound, retinol, resorcinol, a merocyanine compound, mandelic acid, a metal oxalate, a clay, 1,2-heptanediol, 2,3-heptanediol and octyl triazone,

In some embodiments, the composition of the invention is suitable for use as sunscreen lotion, and comprises ingredients selected for improving initial skin feel, skin feel during drying time, and final skin feel.

If the composition of the invention is a cosmetic composition, the composition may optionally include other active or inactive ingredients which are commonly used in cosmetic compositions such as, e.g., cosmetically acceptable carriers, oils, sterols, amino acids, moisturizers, powders, colorants (including pigments and/or dyes) pH adjusters, perfumes, essential oils, cosmetically active ingredients, vitamins, fatty acids, sphingolipids, self-tanning compounds such as dihydroxyacetone (DHA) and erythruloses, excipients, fillers, emulsifying agents, surfactants, film formers, chelating agents, gelling agents, thickeners, emollients, humectants, moisturizers, minerals, viscosity and/or rheology modifiers, keratolytics, retinoids, hormonal compounds, alpha-hydroxy acids, alpha-keto acids, anti-mycobacterial agents, anti-fungal agents, anti-microbials, anti-virals, analgesics, anti-allergenic agents, H1 or H2 antihistamines, anti-inflammatory agents, anti-irritants, anti-neoplastics, immune system boosting agents, immune system suppressing agents, anti-acne agents, anesthetics, antiseptics, insect repellents, skin cooling compounds, skin brightening agents, skin protectants, skin penetration enhancers, lubricants, fragrances, colorants, depigmenting agents, preservatives, stabilizers, pharmaceutical agents, spherical powders and mixtures thereof.

The cosmetic composition of the invention may comprise further optional additives. For instance, one or more fragrances; plant extracts; absorbents; thickeners/rheology modifiers; salicylic acid; alpha- and beta-hydroxy acids; vitamins including vitamins A, C, and E; retinol and its derivatives; preservatives; or any mixtures thereof, may be included in the cosmetic composition.

It may also be advantageous to include one or more tyrosinase inhibitors in the composition of the invention. A specific example thereof is rucinol, also known as 4-n-butylresorcinol. Other examples of suitable tyrosinase inhibitors include thiamidol and other naturally occurring inhibitors such as, e.g., epigallocatechin gallate, quercitin, arbutin, azelaic acid, ellagic acid, kojic acid, hydroquinone, and aloesin.

The present invention further provides a composition of the invention for use in therapy.

The present invention further provides a composition of the invention for use in reducing pigmentation, skin-darkening and erythema caused by solar radiation in melamin-rich skin.

The present invention further provides the use of a composition of the invention in the manufacture of a medicament for reducing pigmentation, skin-darkening and erythema caused by solar radiation in melamin-rich skin.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description making apparent to those of skill in the art how the several forms of the present invention may be embodied in practice.

Two compositions which are suitable for use in the method of present invention were prepared using methods known to those skilled in the art of formulating compositions such as cosmetic compositions. The components of these compositions are listed in Table I below.

In general, all water-soluble ingredients are placed in a main vessel such as thickeners, chelating agents, humectants, pH adjusters, water dispersible film formers, hydrophilic skin adhesion promoters, and/or water-soluble actives. This water phase is mixed until uniform and heated to about 75-80° C.

In a separate vessel, the oil soluble ingredients such as solvents, emollients, emulsifiers, emulsion stabilizers, preservatives, hydrophobic skin adhesion promoters, and/or oil soluble actives are mixed until uniform and heated to about 80° C.

The oil phase is added to the water phase, homogenized through in-line homogenizer to complete 1 cycle for 5 minutes until uniform, and the resultant mixture is cooled to room temperature. During the cooling period, the remaining ingredients are admixed therein. Any ingredients that are heat sensitive including fragrance are added at this point. All raw material is homogenized again through an in-line homogenizer to complete 1 cycle.

The compositions of the present invention such as cosmetic compositions can then be formulated and packaged as a lotion, cream, gel, or other acceptable forms suitable for a cosmetic composition, in particular, a sunscreen composition.

The compositions were subsequently subjected to a clinical study.

TABLE I
Components of tested compositions

| | Ex. 1 | Ex. 2 | Ex. 3 |
|---|---|---|---|
| | Conc. | Conc. | Conc. |
| INCI | (%) | (%) | (%) |
| Aqua + Potassium Lactate + | 0.05 | 0.05 | 0.05 |
| Lactic Acid | | | |
| 4-Butylresorcinol | 0.10 | 0.10 | 0.10 |
| Ethylhexylglycerin | 0.10 | 0.10 | 0.10 |
| Sodium Ascorbyl Phosphate | 0.01 | 0.01 | 0.01 |
| Aqua + Glycerin + PEG-40 Hydrogenated | 0.01 | 0.01 | 0.01 |
| Castor Oil + Trideceth-9 + | | | |
| Phenoxyethanol + Curcuma Aromatica | | | |
| Root Oil + Decylene Glycol + | | | |
| 1,2-Hexanediol | | | |
| Maltodextrin + Citrus Limon | 0.01 | 0.01 | 0.01 |
| Juice + Ascorbic Acid | | | |
| Hydrolyzed Pearl | 0.01 | 0.01 | 0.01 |
| Dibutyl Adipate | 3.00 | 3.00 | 3.00 |
| Isopropyl Palmitate | 2.00 | 2.00 | 2.00 |
| C12-15 Alkyl Benzoate | 4.00 | 4.00 | 4.00 |
| Sodium Cetearyl Sulfate | 0.10 | 0.10 | 0.10 |
| Glyceryl Stearate SE | 2.50 | 2.50 | 2.50 |
| Glyceryl Stearate | 1.45 | 1.45 | 1.45 |
| Diisostearoyl Polyglyceryl-3 Dimer | 0.50 | 0.50 | 0.50 |
| Dilinoleate + C12-15 Alkyl Benzoate | | | |
| Perfume/Fragrance | 0.30 | 0.30 | 0.30 |
| Glycerin + Aqua | 5.00 | 5.00 | 5.00 |
| Citric Acid | 0.06 | 0.06 | 0.06 |
| Phenoxyethanol + Aqua | 0.80 | 0.80 | 0.80 |
| Aqua | 62.80 | 62.20 | 62.30 |
| Alcohol Denat. + Aqua | 2.00 | 2.00 | 2.00 |
| Diethylhexyl Syringylidenemalonate + | 0.00 | 0.50 | 0.00 |
| Caprylic/Capric Triglyceride | | | |
| Pentaerythrityl Tetra-di-t-butyl | 0.00 | 0.00 | 0.50 |
| Hydroxyhydrocinnamate | | | |
| Aqua + Trisodium EDTA | 0.50 | 0.50 | 0.50 |
| Bis-Ethylhexyloxyphenol | 3.00 | 3.00 | 3.00 |
| Methoxyphenyl Triazine | | | |
| Butyl Methoxydibenzoylmethane | 3.00 | 3.00 | 3.00 |
| Ethylhexyl Salicylate | 4.50 | 4.50 | 4.50 |
| Cetearyl Alcohol | 3.00 | 3.00 | 3.00 |
| Xanthan Gum | 0.20 | 0.20 | 0.20 |
| Hydroxypropyl Starch Phosphate | 1.00 | 1.00 | 1.00 |
| Total: | 100.00 | 100.00 | 100.00 |

The composition of Example 1 is a base formula without Pentaerythrityl Tetra-di-t-butyl Hydroxyhydrocinnamate and Diethylhexyl Syringylidene Malonate. The addition of Diethylhexyl Syringylidene Malonate or Pentaerythrityl Tetra-di-t-butyl Hydroxyhydrocinnamate in Example 2 and Example 3 as shown in Table I resulted in protection against skin darkening.

### Description of Clinical Testing

An in vivo efficacy study was conducted on 12 subjects of melanin-rich skin for baseline assessment. Pre-exposure readings were taken before real-life sun exposure, with test products and untreated controls applied at defined doses at time 0. T1 or Immediate Pigment Darkening (IPD) was recorded after 5 minutes of real-life sun exposure. The exposure duration was between 12:00 pm to 2:00 pm. Test products were applied daily on treated sites for seven days by the study coordinator. As a follow-up assessment, T2 or Persistent Pigment Darkening (PPD), was recorded 24-hours after real-life exposure. Finally, T3 was recorded 1-week after real-life exposure.

Subjects of melanin-rich skin were screened and enrolled, followed by initial UV exposure to determine the provisional minimal erythemal dose (MED) during visit one. 24+ hours later, subjects of melanin-rich skin were assessed to determine MED during visit two. During visit 3-5, products were applied to subjects of melanin-rich skin as a pre-treatment for three-days. During visit 6, at T0, pre-exposure readings were taken before UVB, UVA, and VL exposure with test products and untreated controls applied at define doses. T1 was recorded at 5 minutes after solar simulator exposure to determine IPD. The tested composition were then applied on treated sites for seven days by the study coordinator. During visit 7, the PPD was recorded at 24+ hours after UVB, UVA, and VL exposure. Finally, during visit 8, an assessment at +1 week after UVB, UVA, VL exposure was carried out.

### Description of Test Results

TABLE II
Average L* (baseline L* − L* at IPD/PPD/Week1) values of different
sites of subjects' post exposure of real sun light.

| Time points | Ex. 1 | Ex. 2 | Ex. 3 |
|---|---|---|---|
| IPD | −7.12 | −6.29 | −7.12 |
| PPD- 24 h | −5.96 | −4.29 | −4.86 |
| W 1- 1 week | −4.30 | −2.86 | −3.11 |

TABLE III
Effect of compositions on Δ Oxy-Hemoglobin
of sites exposed to real sun exposure.

| Time points | Ex. 1 | Ex. 2 | Ex. 3 |
|---|---|---|---|
| IPD | 0.038 | 0.026 | 0.017 |
| PPD- 24 h | 0.039 | 0.034 | 0.022 |
| W 1- 1 week | 0.012 | 0.001 | −0.01 |

The results shown in Table II illustrate that there is a reduction in skin darkening and pigmentation of melanin-rich skin after real-light exposure caused by the presence of Diethylhexyl Syringylidene Malonate or Pentaerythrityl Tetra-di-t-butyl Hydroxyhydrocinnamate. As shown above, the composition containing only UV filters (Example 1) was found to be inferior to the compositions combining UV filters with Diethylhexyl Syringylidene Malonate (Example 2) and/or Pentaerythrityl Tetra-di-t-butyl Hydroxyhydrocinnamate (Example 3). Further, the compositions of Examples 2 and 3 offer superior protection against IPD and PPD compared to the composition containing only UV filters (Example 1).

In addition, the composition comprising Pentaerythrityl Tetra-di-t-butyl Hydroxyhydrocinnamate (Example 3), demonstrated effective protection against skin darkening when compared to the placebo at IPD.

Further, the composition comprising Pentaerythrityl Tetra-di-t-butyl Hydroxyhydrocinnamate (Example 3) showed significant protection against skin darkening at both PPD and one-week assessments compared to the formula comprising only UV filters (Example 1).

Table III shows the change in oxy-hemoglobin. This change indicates how much erythema is generated even if it is not clearly seen in melanin-rich skin subjects. The duration of exposure was sufficient to cause erythema. As shown in Table III, oxy-hemoglobin levels peak at the 24-hr time point, mirroring the pattern of erythema, which is most prominent during visual examination. While UV filters are generally sufficient for erythema protection, they underperform when compared to compositions that combine UV filters with Diethylhexyl Syringylidene Malonate (Example 2) and/or Pentaerythrityl Tetra-di-t-butyl Hydroxyhydrocinnamate (Example 3). Specifically, the composition containing only UV filters (Example 1) is inferior in controlling oxy-hemoglobin levels independently. In contrast, compositions comprising Diethylhexyl Syringylidene Malonate (Example 2) and/or Pentaerythrityl Tetra-di-t-butyl Hydroxyhydrocinnamate (Example 3), effectively restrict oxy-hemoglobin levels and consequently reduce erythema.

To sum up, the present invention provides:
1. A method of reducing pigmentation, skin-darkening and erythema caused by solar radiation in melanin-rich skin, wherein the method comprises applying to skin in need thereof a cosmetic composition comprising Pentaerythrityl Tetra-di-t-butyl Hydroxyhydrocinnamate and/or Diethylhexyl Syringylidene Malonate in an amount which is effective for reducing pigmentation, skin darkening and erythema caused by solar radiation in melanin-rich skin.
2. The method of item 1, wherein the method is a therapeutic method.
3. The method of item 1 or item 2, wherein the composition comprises at least Pentaerythrityl Tetra-di-t-butyl Hydroxyhydrocinnamate.
4. The method of item 1, wherein the composition comprises at least Diethylhexyl Syringylidene Malonate.
5. The method of item 1, wherein the composition comprises Pentaerythrityl Tetra-di-t-butyl Hydroxyhydrocinnamate and Diethylhexyl Syringylidene Malonate.
6. The method of any one of items 1 to 4, wherein the composition comprises Pentaerythrityl Tetra-di-t-butyl Hydroxyhydrocinnamate and/or Diethylhexyl Syringylidene Malonate in a total concentration of from 0.05% to 2% by weight, based on a total weight of the composition.
7. The method of any one of items 1 to 5, wherein the composition comprises Pentaerythrityl Tetra-di-t-butyl Hydroxyhydrocinnamate in a concentration of from 0.1% to 1% by weight, based on a total weight of the composition.
8. The method of any one of items 1 to 6, wherein the composition comprises Diethylhexyl Syringylidene Malonate in a concentration of from 0.1% to 1% by weight, based on a total weight of the composition.
9. The method of any one of items 1 to 8, wherein the composition further comprises one or more UV filter substances.
10. The method of item 9, wherein the one or more UV filter substances comprise only organic UV filter substances.
11. The method of any one of items 9 and 10, wherein the one or more UV filter substances comprise at least one of Bis-Ethylhexyloxyphenol Methoxyphenyl Triazine, Butyl Methoxydibenzoylmethane, and Ethylhexyl Salicylate.
12. The method of any one of items 9 to 11, wherein the one or more UV filter substances comprise Bis-Ethylhexyloxyphenol Methoxyphenyl Triazine, Butyl Methoxydibenzoylmethane, and Ethylhexyl Salicylate.
13. The method of any one of claims 8 to 11, wherein the composition comprises the one or more UV filter substances in a total concentration of from 1% to 30% by weight, based on a total weight of the composition.
14. The method of item 13, wherein the composition comprises the one or more UV filter substances in a total concentration of from 5% to 15% by weight, based on a total weight of the composition.
15. The method of any one of items 1 to 14, wherein oxy-hemoglobin levels are reduced.
16. A cosmetic composition, wherein the composition comprises Pentaerythrityl Tetra-di-t-butyl Hydroxyhydrocinnamate and/or Diethylhexyl Syringylidene Malonate.
17. The composition of item 16, wherein the composition is suitable for use in the method of any one of items 1 to 15 and wherein Tetra-di-t-butyl Hydroxyhydrocinnamate and/or Diethylhexyl Syringylidene Malonate are present in a concentration which is effective for reducing pigmentation, skin darkening and erythema caused by solar radiation in melanin-rich skin when applied to skin in need thereof.
18. The composition of item 16 or item 17, wherein the composition comprises Pentaerythrityl Tetra-di-t-butyl Hydroxyhydrocinnamate and/or Diethylhexyl Syringylidene Malonate in a total concentration of from 0.05% to 2% by weight, based on a total weight of the composition.
19. The composition of any one of items 16 to 18, wherein the composition further comprises one or more UV filter substances.
20. The composition of item 19, wherein the one or more UV filter substances comprise at least one of Bis-Ethylhexyloxyphenol Methoxyphenyl Triazine, Butyl Methoxydibenzoylmethane, and Ethylhexyl Salicylate.
21. The composition of item 19 or item 20, wherein the composition comprises the one or more UV filter substances in a total concentration of from 1% to 30% by weight, based on a total weight of the composition.
22. The composition of any one of items 16 to 21, wherein the composition is present as one or more of an emulsion, a gel, a cream, a lotion or a spray.

What is claimed is:

1. A method of reducing pigmentation, skin-darkening and erythema caused by solar radiation in melanin-rich skin, wherein the method comprises applying to skin in need thereof a cosmetic composition comprising Pentaerythrityl Tetra-di-t-butyl Hydroxyhydrocinnamate and/or Diethylhexyl Syringylidene Malonate in an amount which is effective for reducing pigmentation, skin darkening and erythema caused by solar radiation in melanin-rich skin.

2. The method of claim 1, wherein the composition comprises at least Pentaerythrityl Tetra-di-t-butyl Hydroxyhydrocinnamate.

3. The method of claim 1, wherein the composition comprises at least Diethylhexyl Syringylidene Malonate.

4. The method of claim 1, wherein the composition comprises Pentaerythrityl Tetra-di-t-butyl Hydroxyhydrocinnamate and Diethylhexyl Syringylidene Malonate.

5. The method of claim 1, wherein the composition comprises Pentaerythrityl Tetra-di-t-butyl Hydroxyhydrocinnamate and/or Diethylhexyl Syringylidene Malonate in a total concentration of from 0.05% to 2% by weight, based on a total weight of the composition.

6. The method of claim 1, wherein the composition comprises Pentaerythrityl Tetra-di-t-butyl Hydroxyhydrocinnamate in a concentration of from 0.1% to 1% by weight, based on a total weight of the composition.

7. The method of claim 1, wherein the composition comprises Diethylhexyl Syringylidene Malonate in a concentration of from 0.1% to 1% by weight, based on a total weight of the composition.

8. The method of claim 1, wherein the composition further comprises one or more UV filter substances.

9. The method of claim 8, wherein the one or more UV filter substances comprise only organic UV filter substances.

10. The method of claim 8, wherein the one or more UV filter substances comprise at least one of Bis-Ethylhexyloxyphenol Methoxyphenyl Triazine, Butyl Methoxydibenzoylmethane, and Ethylhexyl Salicylate.

11. The method of claim 8, wherein the one or more UV filter substances comprise Bis-Ethylhexyloxyphenol Methoxyphenyl Triazine, Butyl Methoxydibenzoylmethane, and Ethylhexyl Salicylate.

12. The method of claim 8, wherein the composition comprises the one or more UV filter substances in a total concentration of from 1% to 20% by weight, based on a total weight of the composition.

13. The method of claim 8, wherein the composition comprises the one or more UV filter substances in a total concentration of from 5% to 15% by weight, based on a total weight of the composition.

14. The method of claim 1, wherein oxy-hemoglobin levels are reduced.

15. A cosmetic composition, wherein the composition comprises Pentaerythrityl Tetra-di-t-butyl Hydroxyhydrocinnamate and/or Diethylhexyl Syringylidene Malonate.

16. The composition of claim 15, wherein the composition comprises Pentaerythrityl Tetra-di-t-butyl Hydroxyhydrocinnamate and/or Diethylhexyl Syringylidene Malonate in a total concentration of from 0.05% to 2% by weight, based on a total weight of the composition.

17. The composition of claim 15, wherein the composition further comprises one or more UV filter substances.

18. The composition of claim 17, wherein the one or more UV filter substances comprise at least one of Bis-Ethylhexyloxyphenol Methoxyphenyl Triazine, Butyl Methoxydibenzoylmethane, and Ethylhexyl Salicylate.

19. The composition of claim 17, wherein the composition comprises the one or more UV filter substances in a total concentration of from 1% to 20% by weight, based on a total weight of the composition.

20. The composition of claim 15, wherein the composition is present as one or more of an emulsion, a gel, a cream, a lotion or a spray.
